(19) United States
(12) Patent Application Publication
(10) Pub. No.: US 2026/0041512 A1
(43) Pub. Date: Feb. 12, 2026

(54) TOOLS FOR ROBOTIC MEDICAL SYSTEMS

(71) Applicant: Remedy Robotics, Inc., San Francisco, CA (US)

(72) Inventors: Jake Anthony Sganga, Mill Valley, CA (US); David James Bell, San Francisco, CA (US)

(21) Appl. No.: 19/295,323

(22) Filed: Aug. 8, 2025

Related U.S. Application Data
(60) Provisional application No. 63/681,382, filed on Aug. 9, 2024.
(60) Provisional application No. 63/681,405, filed on Aug. 9, 2024.
(60) Provisional application No. 63/681,384, filed on Aug. 9, 2024.

(51) Int. Cl. A61B 34/00; A61B 34/30; A61M 25/01; A61M 25/09

(52) U.S. Cl. CPC A61B 34/71; A61B 2034/303; A61M 25/0113; A61M 25/09041

(57) ABSTRACT

A feeder mechanism for advancing or retracting an endovascular tool is described. The feeder mechanism is drivable by outputs of a helm on a robotic medical system. A first input of the feeder mechanism is driven by a first output of the helm to open and close the feeder mechanism so that the endovascular tool can be inserted. A second input of the feeder mechanism is driven by a second output of the helm to cause rollers of the feeder mechanism to advance or retract the endovascular tool.

### PRIORITY APPLICATIONS

This application claims priority to U.S. Provisional Application No. 63/681,382, filed Aug. 9, 2024, U.S. Provisional Application No. 63/681,405, filed Aug. 9, 2024, and U.S. Provisional Application No. 63/681,384, filed Aug. 9, 2024, each of which are incorporated herein by reference. Any and all applications for which a foreign or domestic priority claim is identified in the Application Data Sheet as filed with the present application are hereby incorporated by reference under 37 CFR 1.57.

### BACKGROUND

### Field

Some embodiments of the present disclosure are directed to tools for use with robotic medical systems. In particular, some embodiments of the present disclosure are directed to tools for robotic medical systems configured to facilitate endovascular procedures.

### Description

Endovascular medical procedures are common. During an endovascular procedure, a tool or medical instrument that is generally configured as a thin, flexible, elongate body is inserted into and navigated through a lumen or other cavity of the body.

In some instances, the tools or medical instruments are articulable or controllable, for example, using one or more pull wires, to allow an operator to navigate the tool or medical instrument within the body. Such navigation is often accomplished through deflection (for example, bending) of the distal tip of the tool or medical instrument.

Some tools or medical instruments are configured for manual control, for example, using knobs or levers mounted on a proximally located handle of the tool or medical instrument. In other instances, the tools or medical instruments can be configured for robotic control, for example, control by a robotic medical system. In some embodiments, an operator can use the robotic medical system (for example, a controller, user interface, and/or the like) to robotically control the tool or medical instrument.

### SUMMARY

For purposes of this summary, certain aspects, advantages, and novel features of the invention are described herein. It is to be understood that not all such advantages necessarily may be achieved in accordance with any particular embodiment of the inventions described herein. Thus, for example, those skilled in the art will recognize that the inventions described herein may be embodied or conducted in a manner that achieves one advantage or group of advantages as taught herein without necessarily achieving other advantages as may be taught or suggested herein.

In some aspects, the techniques described herein relate to a feeder mechanism, including: a stationary body; a pivot body pivotally coupled to the stationary body at a pivot; a first roller rotatably mounted to the stationary body; a second roller rotatably mounted to the pivot body; a first input configured to be rotatable by a corresponding first output on a helm, the first input mechanically coupled to the first roller and the second roller such that rotation of the first input causes rotation of the first roller and the second roller; a second input configured to be rotatable by a corresponding second output on the helm, the second input mechanically coupled to the pivot body such that rotation of the second input causes the pivot body to rotate about the pivot relative to the stationary body, wherein: rotation of the second input in a first direction causes the pivot body to pivot to a closed configuration wherein an endovascular tool is clamped between the first roller and the second roller, and rotation of the second input in a second direction causes the pivot body to pivot to an open configuration wherein the endovascular tool can be loaded into or unloaded from the feeder mechanism.

In some aspects, the techniques described herein relate to a feeder mechanism, further including a slot formed in through an upper surface of the feeder mechanism such that the endovascular tool is top loadable into the feeder mechanism.

In some aspects, the techniques described herein relate to a feeder mechanism, wherein the first and second rollers are mechanically coupled to the first input through one or more drive gears.

In some aspects, the techniques described herein relate to the feeder mechanism of Claim of 3, wherein the first and second rollers are rotated in opposite directions.

In some aspects, the techniques described herein relate to a feeder mechanism, wherein the second input is mechanically coupled to the pivot body by a screw mechanism.

In some aspects, the techniques described herein relate to a feeder mechanism, wherein the first input is located on one side of the feeder mechanism and the second input is located on an opposite side of the feeder mechanism.

In some aspects, the techniques described herein relate to a feeder mechanism, wherein the endovascular tool includes a catheter.

In some aspects, the techniques described herein relate to a feeder mechanism, wherein the endovascular tool includes a wire.

In some aspects, the techniques described herein relate to a reel mechanism for a steerable catheter, including: a first input configured to be rotatable by a first output of a helm; a second input configured to be rotatable a second output of a helm; a first pulley, a second pulley, a third pulley, and a fourth pulley positioned at 90-degree intervals around an axis, wherein the axis is aligned with an elongated body of an endovascular tool extending from the reel mechanism; wherein the first input is mechanically coupled to the first pulley and the third pulley, wherein rotation of the first input is configured to cause rotation of the first pulley and the third pulley; wherein the second input is mechanically coupled to second pulley and the fourth pulley, wherein rotation of the second input is configured to cause rotation of the second pulley and the fourth pulley; wherein a first pair of opposing pullwires of the elongated body are wound on the first pulley and the third pulley; and wherein a second pair of opposing pullwires of the elongated body are wound on the second pulley and the fourth pulley.

In some aspects, the techniques described herein relate to a reel mechanism, wherein the first input is mechanically coupled to the first pulley and the second pulley by a first drive gear.

In some aspects, the techniques described herein relate to a reel mechanism, wherein the first drive gear includes a bevel gear.

In some aspects, the techniques described herein relate to a reel mechanism, wherein the second input is mechanically coupled to the second pulley and the fourth pulley by a second drive gear.

In some aspects, the techniques described herein relate to a reel mechanism, wherein the second drive gear includes a bevel gear.

In some aspects, the techniques described herein relate to a reel mechanism, wherein the endovascular tool includes a quad steerable catheter.

In some aspects, the techniques described herein relate to a robot-to-patient (RTP) interface, including: a first body, the first body including a generally cylindrical body extending between a proximal face configured to couple to a helm or a tool positioned in the helm and a distal face, the distal face including a funnel-shaped opening, the first body further including a longitudinal slot; a second body including a semi-cylindrical body, the second body received within a semi-cylindrical groove formed circumferentially in the first body, the second body slidable within the groove, wherein the second body is slidable between: a first position wherein the longitudinal slot is open; and a second position wherein the longitudinal slot is closed.

In some aspects, the techniques described herein relate to a RTP interface, wherein in the open position an endovascular tool can be loaded into the RTP interface such that the endovascular tool extends through the RTP interface.

In some aspects, the techniques described herein relate to a RTP interface, wherein in the closed position the endovascular tool is captured by but is advanceable or retractable through the RTP interface.

In some aspects, the techniques described herein relate to a RTP interface, wherein the second body further includes a tab extending therefrom.

In some aspects, the techniques described herein relate to a RTP interface, wherein the first body further includes a tab extending therefrom.

In some aspects, the techniques described herein relate to a RTP interface, wherein the endovascular tool includes a catheter or a wire.

Although several configurations, examples, and illustrations are disclosed below, it will be understood by those of ordinary skill in the art that the disclosure extends beyond the specifically disclosed configurations, examples, and illustrations and includes other uses of the disclosure. Configurations are described with reference to the accompanying figures, wherein like numerals refer to like elements throughout. The terminology used in the description presented herein is not intended to be interpreted in any limited or restrictive manner simply because it is being used in conjunction with a detailed description of some specific configurations of the disclosure. In addition, configurations can comprise several novel and inventive features. No single feature is solely responsible for its desirable attributes or is essential to practicing the disclosure herein described.

### BRIEF DESCRIPTION OF THE DRAWINGS

Various features will now be described with reference to the following drawings. Throughout the drawings, reference numbers may be re-used to indicate correspondence between referenced elements. The drawings are provided to illustrate examples described herein and are not intended to limit the scope of the disclosure.

FIG. 1 illustrates a perspective view of an embodiment of some components of a robotic medical system that includes an endovascular robot configured to facilitate endovascular procedures.

FIG. 2 illustrates a perspective view of an embodiment of the endovascular robot of the robotic medical system of FIG. 1 shown with no tools attached thereto.

FIG. 3 is a perspective view of an embodiment of a bed mount useable to attach and position an endovascular robot to a bed in the robotic medical system of FIG. 1.

FIGS. 4A and 4B are example fluoroscopic images of a catheter configured with radio-opaque fiducials that allow determination of catheter pose using a computer vision algorithm.

FIG. 5A illustrates how the example arrangement radio-opaque fiducials shown in FIGS. 4A and 4B positioned on the distal end of the catheter may provide a unique appearance at different inclination and roll angles.

FIG. 5B illustrates another example of how the radio-opaque fiducials shown in FIGS. 4A and 4B positioned on the distal end of the catheter may provide a unique appearance at roll angles.

FIG. 6A illustrates examples of projections of three-dimensional generated catheters onto real world two-dimensional X-ray images.

FIGS. 6B-6C illustrate an example prediction of a neural network for predicting the position of a body of a catheter.

FIG. 6D illustrates an example, in which a neural network has identified the catheter within the image, superimposed its estimated centerline onto the image, and highlighted the catheter.

FIG. 6E illustrates an example in which the distal tip position and heading angle have been determined and the image has been updated to include a highlight identifying the position and an arrow indicating the heading.

FIGS. 7A-7D illustrate an embodiment of a graphical user interface for providing image space control of a medical instrument.

FIG. 8 illustrates an embodiment of a user control station that can be used to control the robotic medical system.

FIG. 9A is a perspective view of an embodiment of a feeder mechanism configured for use with a robotic medical system.

FIG. 9B is a top view of the feeder mechanism of FIG. 9A.

FIG. 10A is a perspective view of another embodiment of a feeder mechanism configured for use with a robotic medical system.

FIG. 10B is a front view of the feeder mechanism of FIG. 10A.

FIG. 10C is a cross-sectional view of the feeder mechanism of FIG. 10A.

FIG. 11A is a perspective view of an embodiment of a reel mechanism for a steerable catheter configured for use with a robotic medical system.

FIG. 11B is a top view of the reel mechanism of FIG. 11A.

FIG. 12A is a perspective view illustrating an example schematic arrangement of inputs, drive gears, and reels for a reel mechanism according to an embodiment.

FIG. 12B is a front view of the schematic arrangement of FIG. 12A.

FIG. 13A is a perspective view of an embodiment of a reel mechanism for a steerable catheter configured for use with a robotic medical system. Reel mechanism of FIG. 13A includes an arrangement of inputs, drive gears, and reels as shown in FIGS. 12A and 12B.

FIG. 13B is another perspective view of the reel mechanism of FIG. 13A.

FIG. 14A is a perspective view of an embodiment of a robot-to-patient interface.

FIG. 14B is another perspective view of the robot-to-patient interface of FIG. 14A.

FIG. 14C is a top view of the robot-to-patient interface of FIG. 14A.

FIG. 15A is a perspective view of another embodiment of a robot-to-patient interface.

FIG. 15B is a front view of the robot-to-patient interface of FIG. 15A.

FIG. 15C is another perspective view of the robot-to-patient interface of FIG. 15A.

FIG. 15D is a cross-sectional view of the robot-to-patient interface of FIG. 15A.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without specific details. Further, well-known features may be omitted or simplified in order not to obscure the examples being described.

### Endovascular Procedures

Endovascular procedures are minimally invasive medical techniques used to treat medical conditions that occur inside blood vessels (arteries or veins) using catheters and other tools that are inserted through small incisions, usually in the groin or wrist. In general, an endovascular procedure involves inserting a catheter (e.g., a thin, flexible tube) or other tool into a blood vessel. The catheter or tool is guided through the vasculature to a target area typically using imaging techniques such fluoroscopy (real-time X-ray). Once the catheter or tool is positioned at the target site, tools or other devices (such as balloons, stents, or coils) can be passed through the catheter to treat the issue.

Traditional methods for navigating through the vasculature involve manually advancing and/or rotating a simple catheter over a guidewire into the correct location. Such manual techniques require a high degree of skill and are often imprecise. This lack of precision can result in substantial vessel wall trauma, higher risk of complications, and longer procedure durations that are more difficult to perform.

Common endovascular procedures include, for example, angioplasty (widening narrowed or blocked arteries with a balloon), stenting (placing a small mesh tube (stent) to keep a vessel open), aneurysm repair (placing a stent graft inside an aneurysm to prevent rupture (e.g., endovascular aneurysm repair (EVAR) for abdominal aortic aneurysm)), thrombectomy/embolectomy (removing a blood clot from a vessel), embolization (blocking blood flow to abnormal vessels or tumors), and carotid artery stenting (used to treat narrowing of carotid arteries in the neck (often to prevent stroke)). The systems and methods described herein can be useful in performing these and other endovascular procedures in an improved manner. Endovascular procedures can provide many benefits, including being less invasive than open surgery, requiring smaller incisions and scars, reducing risk of infection, requiring shorter hospital stays, and/or allowing for faster recovery, among others. Endovascular procedures are commonly performed by interventional radiologists, vascular surgeons, or interventional cardiologists, depending on the condition being treated.

Cardiovascular disease (e.g., stroke, heart attack, blood clots, pulmonary embolism, and the like) is the number one cause of death and disability in the United States and around the world. Much of this can be prevented with better access to endovascular intervention (EI). For example, patient outcomes following stroke are directly related to the time it takes to undergo EI. Studies have shown that a person's chance of a return to functional independence drops by greater than 12% for every hour EI is delayed. This is particularly problematic because, due to a lack of expertise, EI is currently only available in about 2% of United States hospitals. The median time to transfer to one of these hospitals is greater than 3 hours. Devastatingly, this means that two thirds of all large vessel occlusion (LVO) stroke victims in the US lack timely access to EI.

LVO is the most significant cause of adult disability in the world. The only proven way to avoid severe post-stroke disability is to undergo an endovascular procedure called mechanical thrombectomy (MT) as quickly as possible. Despite the fact that the facilities required to perform MT are available in over 50% of United States hospitals, due to a lack of specialist expertise, MT is only offered in 2% of hospitals. This means that for patients who present to one of the 98% of hospitals that do not offer MT, treatment is either significantly delayed or not offered at all because timely transfer is not feasible. Because stroke is such a time-sensitive condition, any delay can lead to permanent disabilities or even death.

MT is the current gold standard of care for stroke treatment. MT involves physically retrieving a clot (via stent or suction) from within a blood vessel to restore blood flow in the brain. During MT, devices are threaded through the patient's vasculature starting in the femoral artery (groin) or radial artery (arm). The devices are navigated to the site of the clot using angiography (real-time x-ray images of the blood vessels with the injection of radio-opaque contrast agent).

As noted previously, EIs, like mechanical thrombectomy, are typically performed manually and require an onsite specialist. This leads to the problems associated with access to EI described above. However, endovascular treatment is quite unlike other surgery, which makes remotely supervised and autonomous intervention actually feasible. The systems and methods described here enable stroke patients around the world to undergo mechanical thrombectomy and other endovascular procedures as quickly and as safely as possible. Thus, the systems and methods described herein can improve patient outcomes by increasing access to EI as well as providing EI in a safer manner than manual procedures.

### Robotic Medical System

As described herein, endovascular procedures can be provided using a robotic medical system. Use of a robotic medical system for endovascular procedures can both improve patient access to endovascular procedures and facilitate performance of endovascular procedures in a safe, timely, and effective manner.

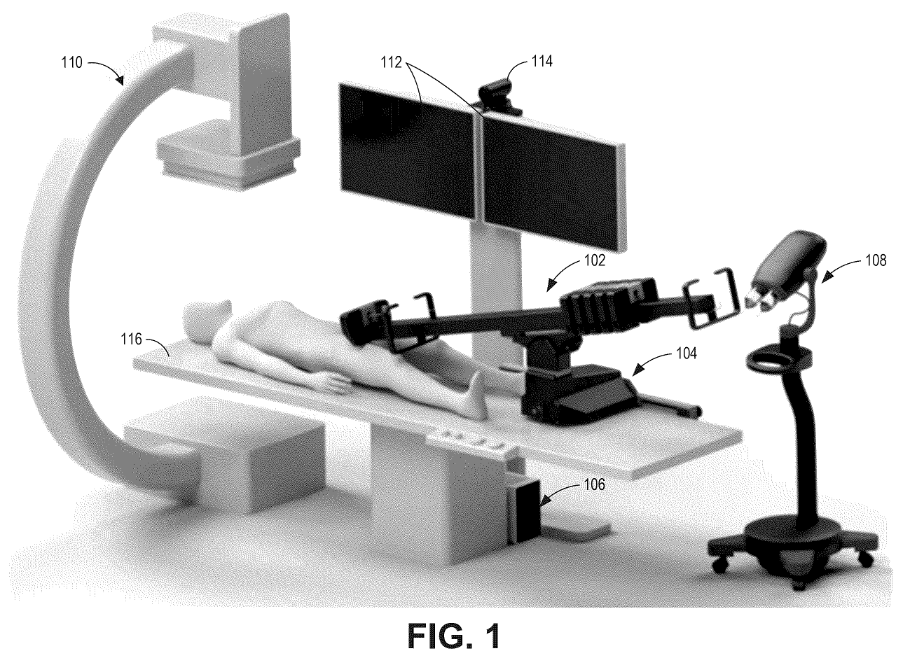

FIG. 1 illustrates a perspective view of an embodiment of some components of a robotic medical system 100. The robotic medical system 100 need not include each and every component shown in FIG. 1, and the robotic medical system 100 may also include other components not illustrated.

The robotic medical system 100 is configured to facilitate a medical procedure on a patient. In some embodiments, the medical procedure is an endovascular procedure. The robotic medical system 100 can include various components including an endovascular robot 102, a bed mount 104, a robot control unit 106, a contrast injector 108, a medical imager 110, one or more displays 112, a camera 114, and a bed 116. The robotic medical system 10 is configured to facilitate performance of an endovascular procedure on a patient positioned on the bed 116. Although not illustrated in FIG. 1, in some embodiments, the robotic medical system 100 may include medical drapes configured to maintain certain components sterile during a procedure.

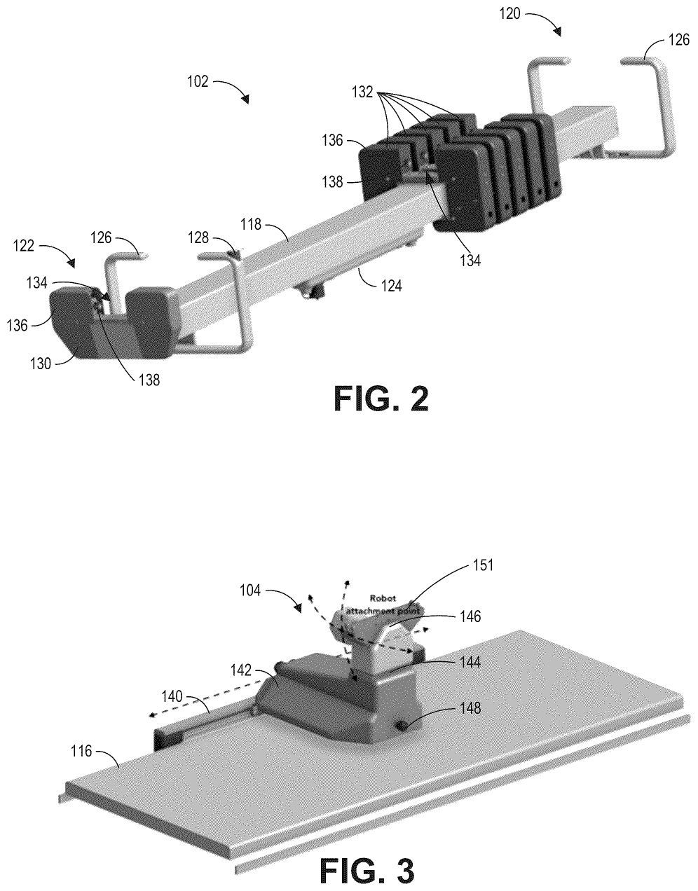

The endovascular robot 102 is shown in greater detail in FIG. 2, which illustrates another perspective view of the endovascular robot 102, shown in an undraped and unmounted position (e.g., not attached to the bed mount 104), illustrated with no tools attached thereto. In some embodiments, for example, as illustrated, the endovascular robot 102 comprises a generally long and slender device. In some embodiments, the endovascular device may comprise a length of about, at least, or at most 150 cm in length, although other lengths both longer and shorter are possible. In some embodiments, the endovascular robot 102 is configured to be lightweight, weighing, for example, less than 40 kg, less than 30 kg, less than 25 kg, or less than 20 kg. Reducing or limiting the weight of the endovascular robot 102 can facilitate installation and movement thereof.

In the illustrated embodiment of FIG. 2, the endovascular robot 102 comprises a rail 118. The rail 118 extends between a proximal end 120 (positioned away from the patient during use) and a distal end 122 (positioned towards the patient during use). The rail 118 can include an attachment device 124 that is configured to secure the endovascular robot 102 to the bed mount 104.

As illustrated in FIG. 2, in some embodiments, the endovascular robot 102 may include one or more handles 126. In the illustrated embodiment, the endovascular robot 102 comprises two handles 126, a first handle 126 positioned at the proximal end 120 of the rail 118, and a second handle 126 positioned at the distal end 122 of the rail 118. The handles 126 can provide locations at which a user (e.g., a medical professional in the operating environment) can grasp the endovascular robot 102 to, for example, reposition it relative to the patient. In some embodiments, during use, the endovascular robot 102 is secured to the bed mount 104 (e.g., see the illustrated configuration of FIG. 1) and the bed mount 104 is configured to provide one or more degrees of freedom that allow the endovascular robot 102 to be repositioned relative to the patient. The bed mount 104 is described in more detail below with reference to FIG. 3. The endovascular robot 102 can include a release button 128, which can be pressed to unlock the endovascular robot 102 and allow it to be repositioned. For example, in some embodiments, the position of the endovascular robot 102 is fixed until the release button 128 is pressed, and while the release button 128 is held, a user can reposition the endovascular robot 102. When the release button 128 is released, the position of the endovascular robot 102 can again be fixed. In the illustrated embodiment, the release button 128 is positioned on the distal handle 126, although other positions for the release button 128 are possible. For example, additionally or alternatively, the release button 128 can be positioned on the proximal handle 126.

In the illustrated embodiment of FIG. 2, the endovascular robot 102 includes a stationary helm 130 and a plurality of movable helms 132 positioned on the rail 118. In the illustrated embodiment, the stationary helm 130 is positioned at the distal end 122 of the rail 118. During use, the endovascular robot 102 is positioned such that the stationary helm 130 is positioned in close proximity to the endovascular access point on the patient (e.g., the patient's groin). The plurality of moveable helms 132 are positioned on the rail 118 and are configured to be independently moveable in proximal and distal directions, along the length of the rail 118. Various mechanical structures can be provided for independently moveable helms 132 along the length of the rail 118. For example, rack and pinion (where a pinion or circular gear within each moveable helm 132 is engaged with a rack or linear gear positioned within the rail 118), screw drives (where a nut within each moveable helm 130 is engaged with a screw positioned within the helm), belt and pulley, and or chain drive systems, among others, can be used for linear movement of the moveable helms 132 along the rail 118.

The stationary helm 130 and each of the plurality of moveable helms 132 can include an attachment location or recess 134 that is configured to receive an endovascular tool therein. The endovascular tools can be removably attachable to the recesses 134 of the stationary helm 130 and moveable helms 132.

Each of the moveable helms 132 can, in some embodiments, include some or all of the following features. The stationary helm 130 can also include some of these features. For example, each helm can include a housing 136. Each helm can include two articulation motors positioned within the housing (not visible in FIG. 2). Each of the articulation motors can be configured to drive a corresponding output 138, which can be configured as an output gear. When a disposable tool is loaded into the recess 134 of the helm, an input on the tool can engage with the output 138 such that the input of the tool can be driven by the articulation motor. Each of the moveable helms 132, can also include an insertion motor positioned within the housing 136 (not visible in FIG. 2). The insertion motor can be configured to cause movement of the moveable helm 132 along the rail. Because the stationary helm 130 is fixed in position, the stationary helm 130 need not include an insertion motor.

In some embodiments, each helm can include a torque sensor (not visible) associated with each motor (e.g., with each articulation motor and with the insertion motor). The torque sensor can be configured to calculate a torque associated with the motor. The torque sensors can be used to measure the force with which the tools are inserted into the patient and actuated. Signals from the torque sensor can be used to, for example, prevent tool insertion beyond a certain force threshold, stop the tool when the insertion force rapidly changes, ensure that the tools are fed into the patient at the same rate as the proximal end of the tool is advanced (in other words, eliminating slack between the feeder and tool), and/or enable precise control of the catheter pull wires associated with the tool.

Each helm can also include an RFID (radio frequency identification) tag reader. In some embodiments, tools that are attachable to the helms can be tracked through an embedded, sterile RFID tag. When a tool is loaded onto a helm, the helm can report the type of tool, as well as other characteristics such as expiration date, manufacturing lot number, tool length, and device-specific calibrations. This tracking ability allows the system to verify that everything is loaded correctly before starting the procedure.

Each helm can also include various electronic components positioned within the housing 136 (e.g., motor controllers, encoders, etc.).

As noted previously and as shown in FIG. 1, the endovascular robot 102 can be mounted to the bed 116 by a bed mount 104. An example embodiment of the bed mount 104 is shown in greater detail in FIG. 3. In the illustrated embodiment, the bed mount 104 is configured to mount to a rail 140 that is positioned on and/or attached to the bed 116. In some embodiments, for example, as illustrated, the rail 140 is positioned on a non-operator side of the bed 116. The bed mount 104 can include a base 142 that is attached to and configured to move linearly along the rail 140. A motor positioned with the base 142 can be configured to drive movement. Such movement can allow linear positioning of the endovascular robot 102 along the bed 116.

The base 142 of the bed mount 104 can include one or more joints (e.g., pivot or swivel joints) that provide additional degrees of freedom for positioning of the endovascular robot. For example, in the illustrated embodiment, the base 142 includes a yaw joint 144 and a pitch joint 146, to allow the yaw and the pitch of the endovascular robot 102 to be adjusted. The bed mount 104 illustrated in FIG. 3, thus allows for three degrees of freedom of adjustment for the endovascular robot 102: linear positioning along the rail 140, and yaw and pitch of the endovascular robot 102. These degrees of freedom are illustrated with dashed line arrows in FIG. 3. In other embodiments, other degrees of freedom can be provided by the bed mount 104 and/or one or more of the illustrated degrees of freedom can be omitted.

In some embodiments, during use, the bed mount 104 is configured so that the endovascular robot 102 can be positioned over the patient's legs (e.g., as shown in FIG. 1). Thus, the bed mount 104 can allow the distal end 122 of the endovascular robot 102 to be positioned in close proximity to an access point (e.g., a femoral sheath) positioned at the patient's groin.

In some embodiments, the bed mount 104 is configured such that, by default, the bed mount 104 is securely locked (e.g., the position is fixed). However, the position can be adjusted by pressing down on the release button 128 described above. That is, in some embodiments, the release button 128 can be held down in order to allow the position endovascular robot 102 to be adjusted. In some embodiments, the bed mount 104 includes one or more spring dampers that ensure that when the bed mount 104 is unlocked, the position of the endovascular robot 102 moves slowly and in a controlled manner to the desired position. Once the robot is correctly positioned, the operator can release the release button 128, and the bed mount 104 will automatically lock.

The bed mount 104 also includes a robot attachment point 151 that is configured to secure to the attachment device 124 on the endovascular robot 102 to secure the endovascular robot 102 to the bed mount 104.

The bed mount 104 can also include an emergency stop switch 148 that can be activated to stop all movement of the bed mount 104, the endovascular robot 102, and/or any tools attached thereto. As shown in FIG. 3, in some embodiments, the emergency stop switch 148 can be located on the front of the bed mount 104 to provide easy and unrestricted access to a bedside operator.

Additional detail regarding bed mounts that can be used with the robotic medical system 100 are described in U.S. application Ser. No. 19/049,670, filed Feb. 10, 2025, which is incorporated herein by reference.

For the robotic medical system 100, various tools can be configured to couple to the helms (e.g., the stationary helm 130 and the moveable helms 132). These tools can be configured to removably attach to the recesses 134 of the helms. In some embodiments, the tools are disposable, such that that are useable for a single procedure and the disposed of. Various tools can be used, including for example, one or more tools configured to linearly advance or retract an elongate device (e.g., a catheter or a guidewire), various catheters (both steerable and non-steerable, various guidewires, and the like. In some embodiments, the tools can include an RFID tag allows the helm to recognize which tool has been loaded onto the helm and whether or not it has been correctly loaded.

In some embodiments, endovascular tools (such as catheters) of the robotic medical system 100 can be configured to include one or more radio-opaque fiducials positioned thereon that are configured to be used in combination with computer vision to infer the pose of the catheter. In some instances, the term “pose” is used herein to refer to the position and orientation of a catheter. In some embodiments, determination of pose can be made based on a two-dimensional medical image, such as a single plane X-ray image, and one or more radio-opaque markers included on a catheter. Computer vision models can be employed to detect the catheter and/or radio-opaque markers positioned thereon in the two-dimensional medical image and to determine the pose of the catheter therefrom. In some instances, the pose can be defined by five degrees of freedom for the catheter. The five degrees of freedom can include two positional degrees of freedom (e.g., x and y position) and three degrees of freedom relating to orientation (e.g., heading, incline, and roll).

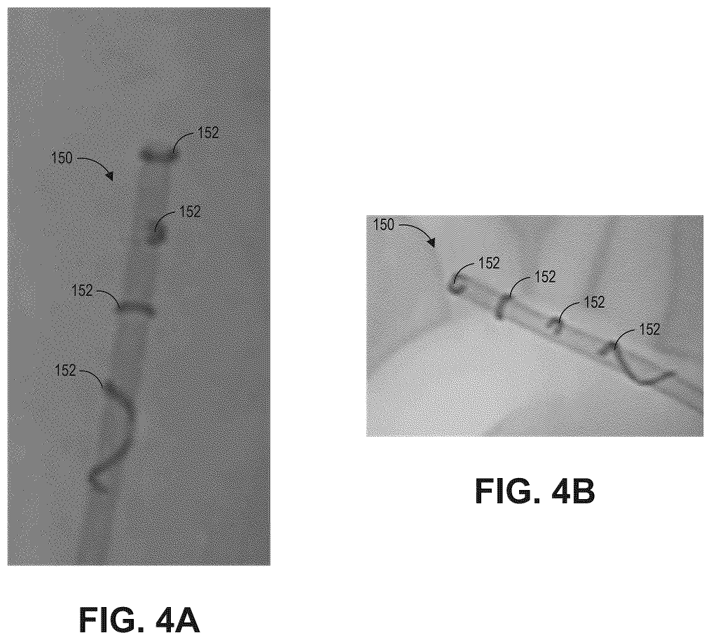

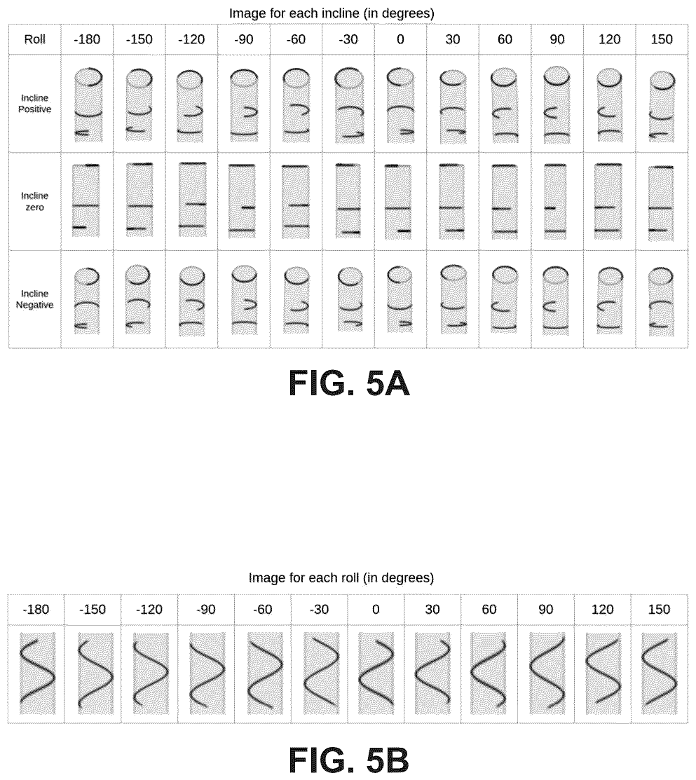

FIGS. 4A-5B illustrate how incline and roll of the catheter can be determined based on the two-dimensional appearance of the radio-opaque markers included on the catheter. Roll is measured around a longitudinal axis of the catheter. Incline is measured relative to the plane of the medical image (e.g., whether the catheter is inclined into or out of the plane of the medical image). FIGS. 4A and 4B, for example, are fluoroscopic images of a catheter 150 configured with radio-opaque fiducials 152 that allow determination of catheter pose using a computer vision algorithm. In the illustrated embodiment, the radio-opaque fiducials 152 comprise three the non-circumferential rings that are semi-circular, extending part way around the catheter 150. In some embodiments, the non-circumferential rings 152 can be radio-opaque such that it can easily be identifiable within a medical image. The appearance of the non-circumferential rings 152 in a two-dimensional medical image can be analyzed, using computer vision to determine the sign and magnitude of the incline of the catheter 150. The sign and magnitude of the incline of catheter 150 can be determined by the unique appearance of the non-circumferential rings 152 in the two-dimensional image at varying degrees of incline, both positive and negative. In some embodiments, non-circumferential rings 152 are arranged in an asymmetrical design. That is, in some embodiments, the non-circumferential rings 152 are each positioned at a different rotational position around the catheter 150. In the illustrated embodiments, the non-circumferential rings are positioned at 90-degree offsets. In some embodiments, non-circumferential rings 152 are multiple ellipses offset from each other.

FIG. 5A illustrates how the example arrangement of non-circumferential rings 152 shown in FIGS. 4A and 4B positioned on the distal end of the catheter 152 may provide a unique appearance at different inclination and roll angles. Images are provided at positive, neutral (i.e., zero), and negative inclinations, as well as at different roll positions provided in 30-degree increments. As shown, each of the 36 different illustrated positions provides a unique appearance. By detecting, for example, using computer vision, this appearance within a medical image, the incline (including its sign) and the roll of the catheter can be determined. While FIG. 5A illustrates how the radio-opaque markers 152 provide different two-dimensional appearances for different roll positions at 30-degree increments and for different positive, neutral (zero), and negative inclines, the illustrated increments are not intended to be limiting.

Returning to FIGS. 4A and 4B, the illustrated embodiment of the catheter 150 also includes a different radio-opaque fiducial 152 having a helical shape. FIG. 5B illustrates the two-dimensional appearance (e.g., as within the plane of medial image) of the helical fiducial 152 of FIGS. 4A and 4B and different roll positions in 30-degree increments. As shown, each roll position provides a unique appearance which can be used to determine roll, for example, by a computer vision, neural network, or machine learning system. While FIG. 5B illustrates how the radio-opaque markers provide different two-dimensional appearances for different roll positions at 30-degree increments, the illustrated increments are not intended to be limiting.

In some embodiments, a computer system may utilize artificial intelligence or machine learning to perform such functionality. In some embodiments, for example, a neural network can be trained to detect the position/appearance of the radio-opaque fiducials in combination within a two-dimensional image, and extract or determine the incline and/or roll from the detected appearance position. It should be noted that in some embodiments, the machine learning algorithm does not hardcode the aforementioned approach. Instead, the machine learning algorithm trains a deep neural network to directly predict the incline angle or roll from the input of the X-ray image.

Other methods and mechanisms can be used for determining the roll and incline of a catheter in the robotic medical system 100. Further, for embodiments that include radio-opaque fiducials, other configurations for the radio-opaque fiducials can be used.

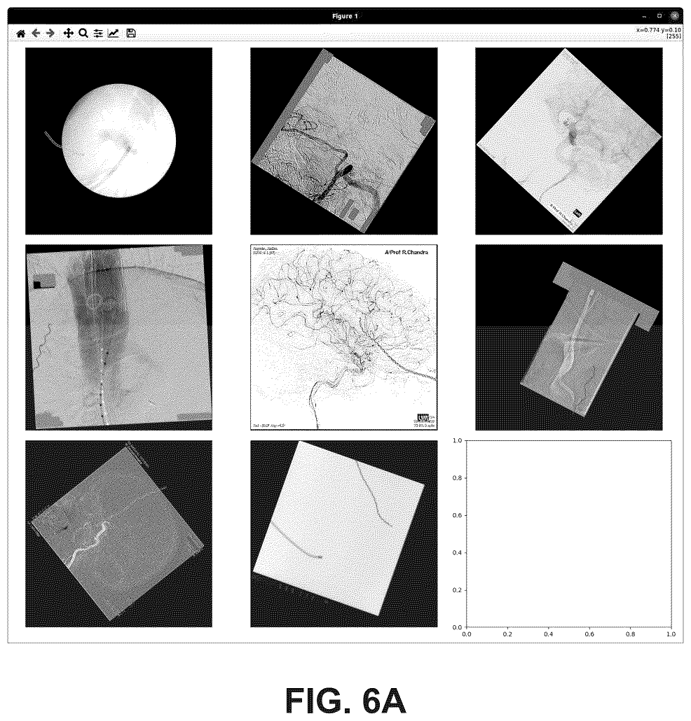

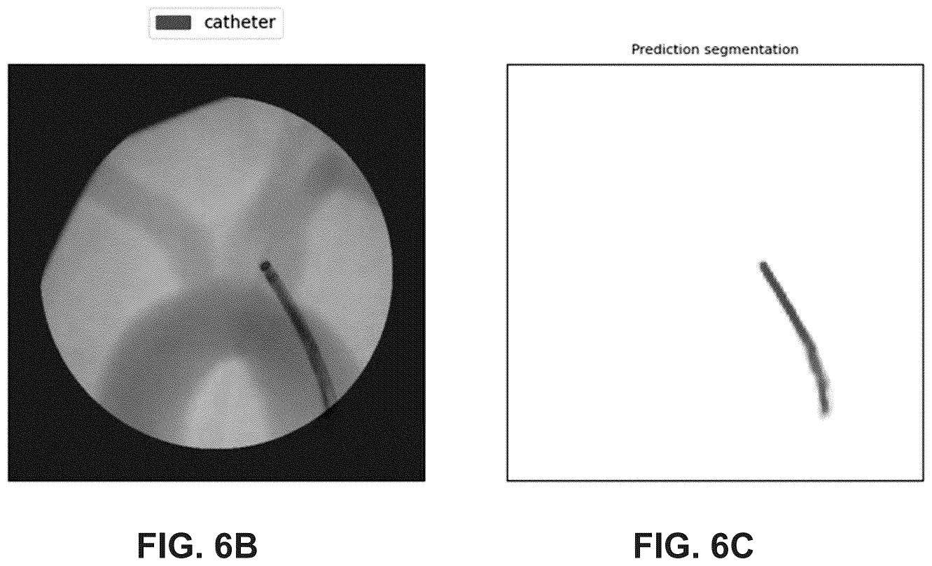

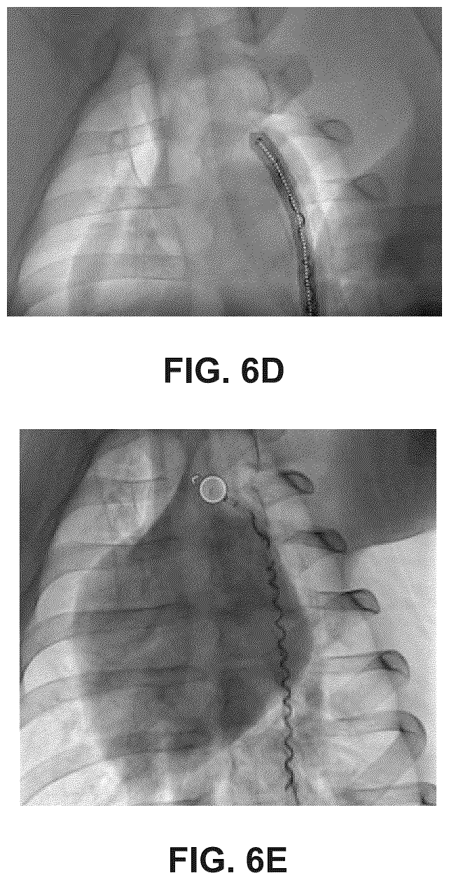

Machine learning and/or computer vision can also be used to determine position (e.g., x, y position) and heading (e.g., direction within the plane of the medical image) of a catheter. FIG. 6A illustrates examples of projections of three-dimensional generated catheters onto real world two-dimensional X-ray images. FIGS. 6B-6C illustrate an example prediction of the trained deep neural network for predicting where the catheter body is. As noted above, position can refer to translation of endovascular and/or other intraluminal tools along the x, y directions in the plane of the medical image. In some embodiments, the system may be configured to predict where the full tool body is, and then from this tool body, the two-dimensional tip location can be extracted. This approach may be beneficial because the tool body provides a very strong training signal for learning deep neural network segmentation models. That is, in some instances, it may be easier for a neural network of computer vision algorithm to detect the body of a catheter and then extract the location of the tip from there. In some embodiments, catheter kinematics are further used to refine this estimate.

For example, a deep neural network can be used to estimate the two-dimensional centerline position of the catheter based on one or more images of the catheter navigating within the body. FIG. 6D illustrates an example, in which the neural network has identified the catheter within the image, superimposed its estimated centerline onto the image, and highlighted the catheter. Once the centerline of the catheter has been identified within the image, the two-dimensional position can be directly extracted by computing the most distal position along the centerline. Similarly, the heading of the catheter can also be directly extracted from the body estimate by computing the vector of the tip of the body line. FIG. 6E illustrates an example in which the distal tip position and heading angle have been determined and the image has been updated to include a highlight identifying the position and an arrow indicating the heading.

In some embodiments, a machine learning algorithm for estimating the position of a catheter and/or other tool may use the following approach. First, the image generation procedure is modified by drawing the catheter on top of the medical image (e.g., as shown in FIG. 6B). This process may have the advantage of training the deep neural network with realistic noise and occlusions that would be seen in actual X-rays, making the system robust to real world conditions.

Second, the two-dimensional x and y position is estimated. In some embodiments, radio-opaque markings may be added to the tool body, such as, for example, a full-length helix, to assist with the identification. Thereafter, the two-dimensional x and y position location of the tool tip can be determined. This approach may be used because the tool body provides a very strong training signal for learning deep neural network segmentation models. In some embodiments, catheter kinematics may be used to further refine the position estimate.

To determine the heading of endovascular and/or other intraluminal tools, such as a catheter, the deep neural network prediction of the catheter body position may be used. Based on the prediction of the two-dimensional x and y position of the catheter tip, a second position located on the catheter body may be determined. The second position may be an infinitesimal distance from the tool tip in a direction along the catheter body. The heading angle of the catheter may then be calculated using trigonometry based on the x and y position of the tool tip and the second position along the catheter body.

Additional detail regarding pose determination is described in U.S. application Ser. No. 17/819,101, filed Aug. 11, 2022, and granted as U.S. Pat. No. 11,707,332 on Jul. 25, 2023, each of which is incorporated by reference herein in its entirety. Other methods and mechanisms for determining position and heading of the catheter may also be used.

Thus, in some embodiments of the robotic medical system 100, computer vision can be used to infer tool position, orientation, incline and roll, we solve this problem and make catheter control simple. That is, computer vision can analyze a medical image to determine the tool position (e.g., as represented by a two-dimensional line corresponding to the centerline of the catheter), tool tip position (e.g., as represented as an x, y coordinate in the plane of the medical image); tool tip heading (e.g., the direction the catheter is pointing in the image plane), tool incline (e.g., to the degree to which the tip is pointing up towards the x-ray imager or down away from the x-ray imager), and/or tool roll (e.g., how the catheter is rotated about itself).

By understanding all of these position components of the tool, the robotic medical system can be configured to control the catheter autonomously or, at the very least, make control for the user extremely simple.

For example, in some embodiments, the robotic medical system 100 can be configured to allow for “image space control” wherein control inputs are provided with respect to how the tool appears in the medical image, regardless of the current orientation of the tool. This type of control system is intuitive as the user may provide such inputs while viewing the medical image which includes at least a representation of a distal portion of the instrument. That is, the user can provide control inputs relative to the current appearance of the instrument within a medical image. In this way, the user can provide natural and intuitive inputs with respect to the current position and orientation of the instrument within a medical image, and the system can determine appropriate motor commands (e.g., commands for actuating one or more of the pullwires of the instrument) to cause the desired motion. In some embodiments, this can allow the user to control the catheter in one or more of the following three directions: forward and back (insertion), left and right (heading), and/or into and out of the image (incline). These directions move with respect to the plane of the image regardless of how the X-ray is moved or how the catheter is rolled in the body. This control mode is intuitive and provides a large advancement over the current standard of care, which requires the user to frequently guess and check which way the catheter will move on screen. Using these controls, the user can easily access tricky vessels and ensure safe navigation of the instrument through the vessels in an atraumatic fashion.

A user may provide user inputs in various ways. For example, in some embodiments, the user can specify desired targets for insertion, heading, and/or incline. Once specified, the system can determine the appropriate motor commands for causing the instrument to move from its current position and orientation to the desired position and orientation. Providing such absolute targets (e.g., desired targets for insertion, heading, and/or incline) may advantageously provide some resiliency and safety in the event in a lag in communication between the user and the robotic medical system. This can be advantageous for situations wherein the user is remotely located from the robotic system and patient and communication occurs of a computer network, such as the internet.

As another example, a user may provide user inputs that are indicated relative to the current position or orientation of the instrument. For example, a user can specify that the instrument adjust its heading to the right relative to the current heading of the instrument. While such a system may be less tolerant to high latency and communication lag, it still allows user to navigate in a simple and intuitive manner.

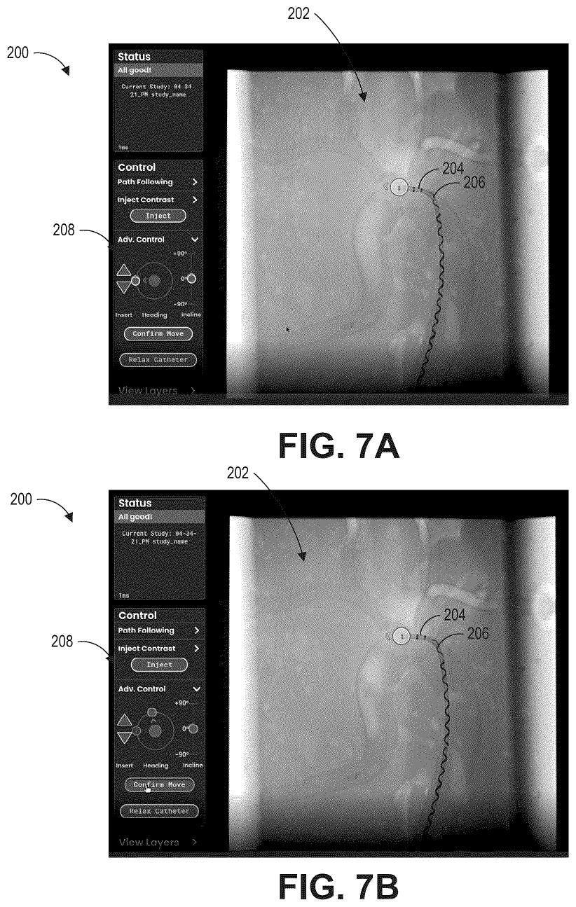

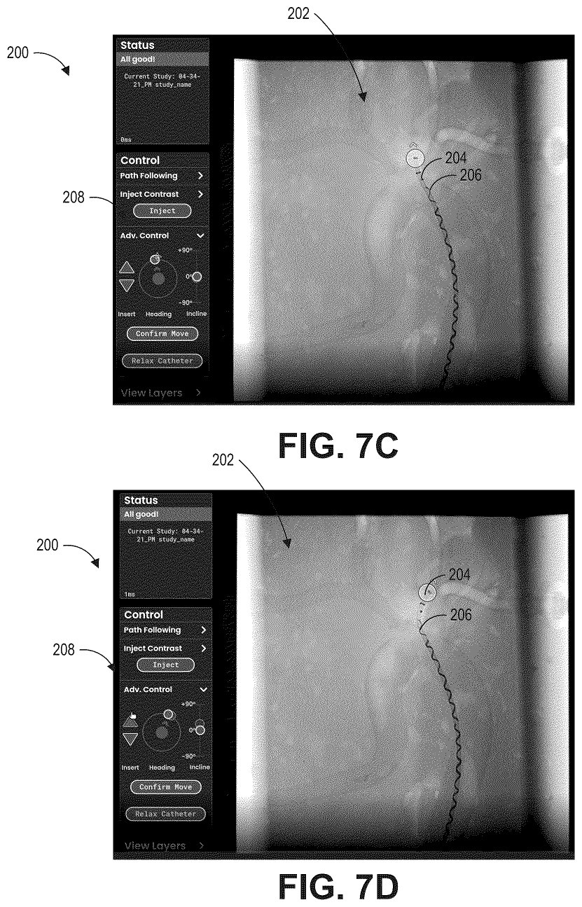

FIGS. 7A-7D illustrate an embodiment of a graphical user interface 200 for providing image space control of a medical instrument. In the illustrated embodiment, the graphical user interface is configured to display a two-dimensional medical image 202, such as an X-ray. The medical image 202 includes a view of a distal end of a medical instrument, such as a catheter 204. The catheter 204 includes one or more fiducials 206 positioned thereon. The fiducials 206 are visible within the medical image 202. The fiducials 206 can be configured as described above in order to allow for vision-based determination of the position and orientation (including roll) of the medical instrument. For example, at least one fiducial 206 can be configured such that it provides unique two-dimensional appearances associated with different roll angles for the catheter 204, for example, as described above with reference to FIGS. 4A-5B.

The graphical user interface 200 may also include a user input device 208. The user input device 208 is configured to receive user inputs from a user that are provided with respect to the two-dimensional medical image 202. For example, in the illustrated embodiment, the user input device 208 includes features for allowing a user to input insert commands (e.g., to advance or retract the instrument 204), heading commands (e.g., to alter the heading of the medical instrument 204 within the plane of the medical image 202, for example, to the right or left of the instrument's current heading), and incline commands (e.g., to alter the incline of the medical instrument 204 into or out of the plane of the medical image 202. The user input device 208 may include other options as well. For example, in the illustrated embodiment, the user input device 208 includes options to inject contrast, confirm an entered movement, and to relax the catheter.

Although the user input device 208 is illustrated as a component of the graphical user interface 200, this need not be the case in all embodiments. For example, in some embodiments, the user input device 208 can comprise a handheld control.

Importantly, the user input device 208 allows the user to provide user inputs for controlling the instrument 204 with respect to the current configuration of instrument as shown in the two-dimensional medical image 202. For example, as shown in FIG. 7B, the user may input a desired heading for the medical instrument via the heading input of the user input device. In the illustrated configuration, the user can input a desired heading by selecting a target point on the wheel. In FIG. 7B, the desired heading is shown at about 355 degrees with a highlighted circle. The current heading is also shown on the wheel at about 270 degrees as a lighter circle. The user may also select a desired inclination using the incline slider, if desired.

Continuing this example, with reference to FIG. 7C, by selecting the confirm move option, the robotic system can determine appropriate motor commands to cause the instrument 204 to move to the desired heading and incline. FIG. 7C shows the instrument 204 after movement. FIG. 7D illustrates that, by using the insert arrows of the user input device 208 the user can command forward and backward motion of the instrument 204.

The graphical user interface 200 and user input device 208 of FIGS. 7A-7D provide only one example of how these features may be configured.

In order to generate appropriate motor commands based on the user inputs to cause the instrument to move appropriately, it is important that the current roll of the instrument be accounted for. This is necessary to ensure that the appropriate pullwires are actuated to cause the specified motion. In some embodiments, the system determines the roll of the instrument automatically, for example, using computer vision analysis of the appearance of one or more of the fiducials in the image as discussed above.

Returning to FIG. 1, the robotic medical system 100 may also include a robot control unit 106, a contrast injector 108, a medical imager 110, one or more displays 112, and a camera 114.

The robot control unit 106 may be a computerized device including a processor and computer memory storing instructions that configure the process to facilitate control of the robotic medical system. For example, the robot control unit 106 unit may facilitate determination of the pose of a medical instrument by analyzing a medical image provided by the medical imager 110. Similarly, the robot control unit 106 may facilitate image space control by translating image space control user inputs into appropriate motor commands for moving a medical instrument.

The contrast injector 108 can be configured to supply contrast to the robotic medical system such that contrast can be injected to facilitate visualization of the vasculature. In some embodiments, the contrast injector 108 comprises a dual headed system, where one head is loaded with radiopaque contrast and the other head is loaded with saline. In other embodiments, the contrast injector can comprise a single headed system, with one head loaded with radiopaque contrast and a heparinized saline flush is hung. The lines can be connected and drain into a single common line which is attached to a connector on the back of a steerable catheter before being flushed and checked that they are free of air.

The medical imager 110 can comprise an X-ray device, such as a C-arm. The medical imager 110 is configured to capture medical images (e.g., X-rays) of the patient during a medical procedure.

Displays 112 can be provided in the operating environment for visualization by medical personnel located onsite. A camera 114 can be positioned to capture an image of the operating environment to, for example, allow a remote operator to visualize the procedure.

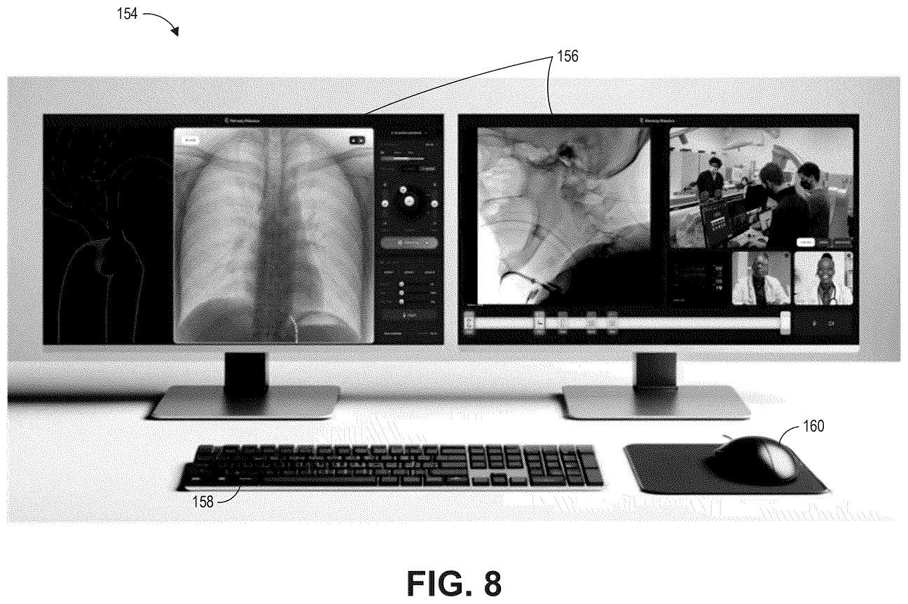

The robotic medical system 100 can be configured to be controllable using computerized equipment, such as a personal computer, a laptop, or a tablet. An example user control station 154 is shown in FIG. 8. This user control station 154 is provided only by way of example, and the user control station 154 may be provided in other forms as well.

In the illustrated embodiment, the user control station 154 comprises a personal computer (not shown), two displays 156, a camera and microphone (not shown), a keyboard 158, and a mouse 160. In some embodiments, the user control station 154 may also comprise a foot pedal (not shown).

In the illustrated embodiment, the user control station 154 is configured to display a graphical user interface. In the illustrated embodiment, left display 156 is configured to display a three-dimensional model of the vasculature of the patient, which can be rotated and manipulated by the user. The left display 156 is also configured to display an X-ray feed, showing an image from the medical imager 110. In the illustrated example, the x-ray feed occupies the majority of screen space on the left display 156. A “live” icon indicates when the X-ray image displayed is a live image. In some embodiments, a co-registered model of the vessels can be overlaid on the X-ray image. The co-registered model can be toggled on and off. The left display 156 can also include user controls for selecting which tools to control, injecting contrast, and moving the tools. In some embodiments, the left display 156 can include image space control user inputs.

In the illustrated embodiment, the right display 156 is configured to display an X-ray feed for biplane imaging, a toggleable camera views directly into the Cath lab (e.g., a view from camera 114), displays of remote viewers and hemodynamics, and a schematic view of the endovascular robot 102 to show which tools are active and show where the helms are in reference to one another.

The illustrated graphical user interface is provided as only one example. Other embodiments can include some, none, or other features. Further, the features on the graphical user interface can be arranged differently. In some embodiments, the graphical user interface can be displayed on a single display so as to be useable on a tablet, smartphone, or laptop. In some embodiments, the user control station 154 can be configured for touchscreen inputs.

Notably, the user control station 154 can be remotely located from the remainder of the robotic medical system 100 components shown in FIG. 1 and can communicate over a network, including the internet. In this way, a remotely located operator can control the robotic medical system 100.

The robotic medical system 100 can be configured to enable a local or remote operator to navigate catheters from the femoral or radial artery or vein to the proximal cerebral, distal cerebral, coronary, pulmonary, aorta, peripheral arteries or veins as well as inject contrast and aspirate a thrombus, deliver a stent retriever, deploy a coil, deploy a stent, and/or deliver a liquid embolic, among other features and procedures.

### Feeder Mechanisms for Endovascular Instruments

This section describes feeder mechanisms for use with robotic medical systems, such as the robotic medical system 100 described above. The feeder mechanisms can be configured as tools that are configured to removably couple to the stationary helm 130 and/or any of the moveably helms 132. As will be described in more detail below, the feeder mechanisms can include inputs that are configured to engage with and be driven by the outputs 138 of the helms 130, 132 (see FIG. 2) to cause the feeder mechanisms to perform various functions.

In general, a feeder mechanism is configured to be received within the recess 134 of a stationary helm 130 or a moveable helm 132. The feeder mechanism is further configured to receive an endovascular tool (e.g., a catheter, microcatheter, or wire) that extends through the feeder mechanism in a proximal-distal direction. As will be described in more detail below, the feeder mechanism includes at least a pair of opposed rollers. The pair of opposed rollers are configured to clamp onto the elongated body of the endovascular tool. One or both of the rollers in the pair is configured to be driven. The rollers can be configured to rotate in opposite directions. That is, as one roller rotates in a clockwise direction, the other roller rotates in a counterclockwise direction and vice versa. The rollers clamp onto and frictionally engage with the elongated body of the endovascular tool passing through the rollers. As the rollers rotate in one direction, the feeder mechanism causes the endovascular body to advance or insert in the distal direction. As the rollers rotate in the opposite direction, the feeder mechanism causes the endovascular body to retract in the proximal direction.

In some embodiments, each roller in a pair of rollers is actively driven. Driving both rollers in a pair may provide for better pushability of the endovascular tool. In other embodiments, only one roller of the pair of rollers may be driven. The other roller in the pair may passively rotate. In some embodiments, the rollers comprise a soft, biocompatible material that provides good friction and engagement between the rollers and the endovascular tool while limiting or reducing damage to the endovascular tool. For example, in some embodiments, the rollers comprise silicon.

In some embodiments, the rollers can be moved between an open position that is used to insert the endovascular tool into the feeder mechanism, and a close position wherein the rollers clamp on and can drive insertion and retraction of the endovascular tool. In some embodiments, moving between the open position and the close position can be caused by one of the outputs 138 on the helm 130, 132.

In some embodiments, the rollers can be isolated from the drive gears and inputs to reduce or eliminate particulate transfer which could increase the likelihood of damage occurring to the endovascular input and/or introduce contaminants.

The feeder mechanisms can be configured to accept a range endovascular tool sizes, for example, tools having diameters from 0 to 3 mm or 0 to 5 mm.

In some embodiments, the feeder mechanisms can include an RFID tag storing information about the feeder mechanism. When the feeder mechanism is coupled to a helm 130, 132, the RFID reader on the helm 130, 132 can read information from the feeder mechanism to identify and correctly control the feeder mechanism.

In some embodiments, the feeder mechanism is sterilizable. In some embodiments, the feeder mechanism is single use and/or disposable.

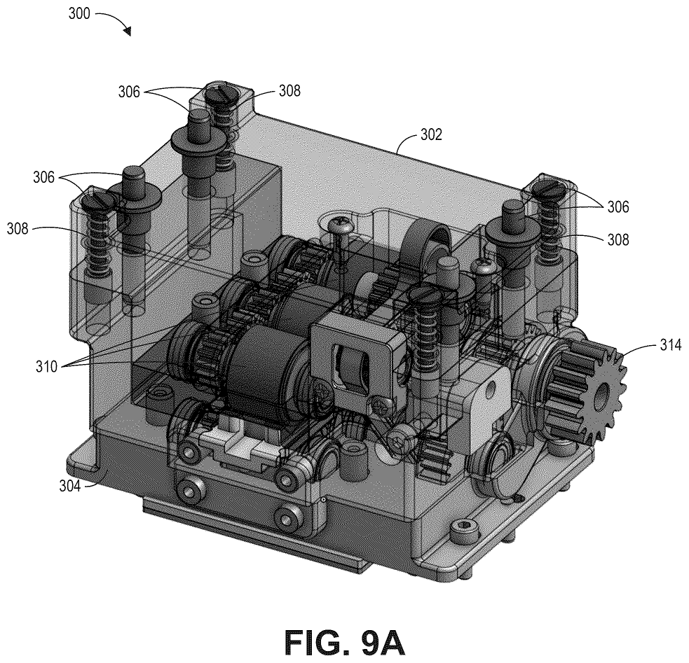

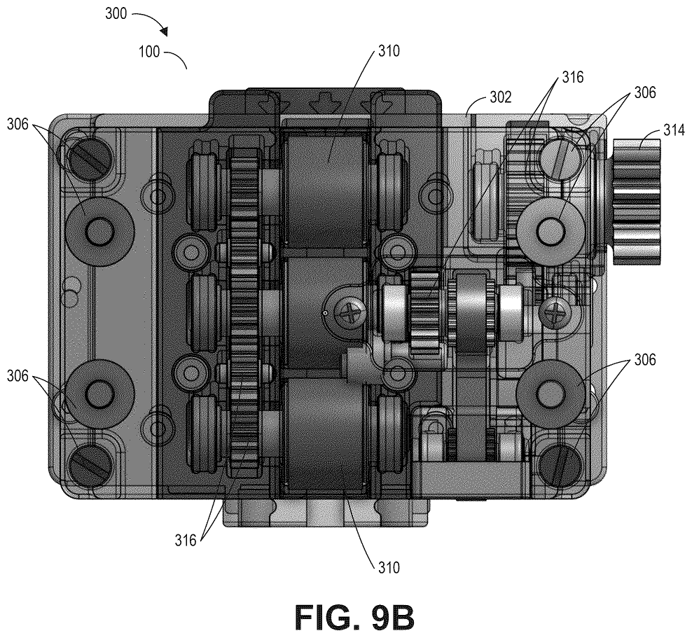

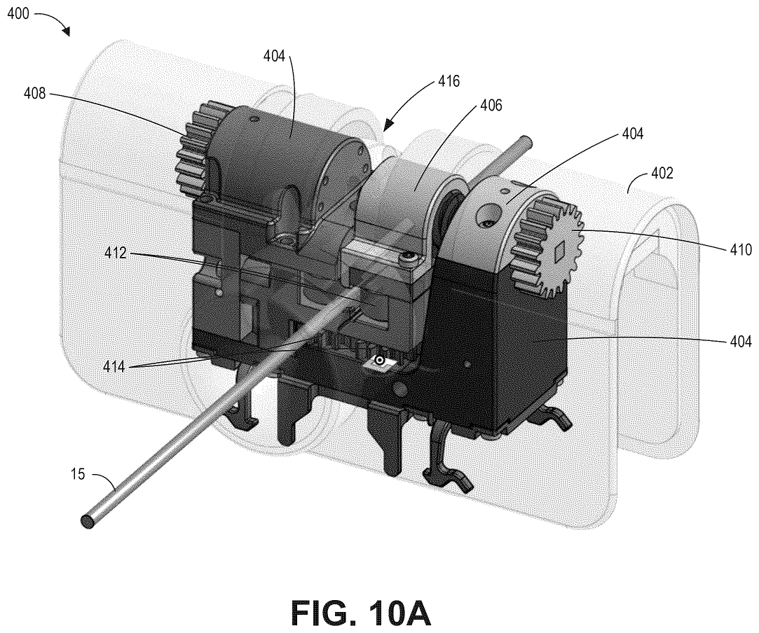

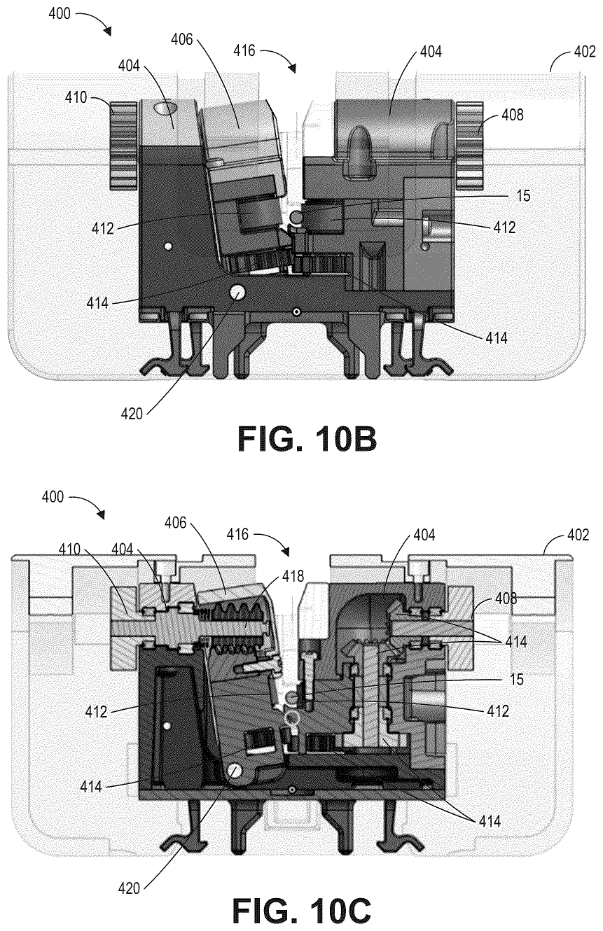

FIGS. 9A and 9B illustrate one embodiment of a feeder mechanism 300. FIGS. 10A-10C illustrate another embodiment of a feeder mechanism 400. Each of the feeder mechanism 300 and the feeder mechanism 400 can include any or all of the features described above, except when noted differently in the corresponding description.

Feeder mechanism 300 will now be described with reference to FIGS. 9A and 9B. FIG. 9A is a perspective view and FIG. 9B is a top view of the feeder mechanism 300. In each of FIGS. 9A and 9B, an upper body 302 of the feeder mechanism 300 is illustrated as transparent so that internal structures of the feeder mechanism 300 can be seen.

The feeder mechanism 300 includes an upper body 302 and a lower body 304. The upper body 302 is slidably mounted on posts 306 which extend upwardly from the upper body. Springs 308 can be included on some of the posts 306 to bias the upper body 302 into contact with the lower body 304.

In the illustrated embodiment, three upper rollers 310 are rotatably mounted in the upper body 302. Similarly, three lower rollers 312 are rotatably mounted in the lower body 304. During use of the feeder mechanism 300, the force of the springs 308 biases the upper body 302 towards the lower body 304 such that an endovascular instrument passing through the feeder mechanism 300 is clamped between the upper rollers 310 and the lower rollers 312. Although the illustrated embodiment includes three upper rollers 310 and three lower rollers 312, other numbers of upper and lower rollers can be used. For example, the feeder mechanism 300 can include one, two, three, four, or more upper rollers 310 and corresponding lower rollers 312.

To insert the endovascular tool into the feeder mechanism 300, a user may grasp the upper body 302, pulling it upward overcoming the bias of the springs 308 to cause the upper body 302 to separate from the lower body 304. The endovascular tool can then be fed between the upper rollers 310 and the lower rollers 312. The upper body 302 can then be released, causing the upper body 302 to move towards the lower body 304, thereby clamping the endovascular tool between the upper rollers 310 and the lower rollers 312.

The feeder mechanism 300 includes an input 314. In the illustrated embodiment, the input 314 comprises a gear. When the feeder mechanism 300 is inserted into the recess 134 of a helm 130, 132, the output 138 of the helm 130, 132 engages with the input 314 of the feeder mechanism 300. A motor in the helm 130, 132 can thereby drive the input 314 through the output 138. In the illustrated embodiment of the feeder mechanism 300, the feeder mechanism 300 includes only a single input 314. The single input 314 can be configured to cause rotation of the upper rollers 310 and/or the lower rollers 312. For example, a plurality of drive gears 316 can couple the input 314 to the upper rollers 310 and/or the lower rollers 312.

The feeder mechanism 400 will now be described with reference to FIGS. 10A-10C. FIG. 10A is a perspective view, FIG. 10B is a front view, and FIG. 10C is a cross-sectional view of the feeder mechanism 400. In FIGS. 10A and 10B a cover 402 of the feeder mechanism 400 is illustrated as transparent to allow visualization of the internal components of the feeder mechanism.

As shown in FIGS. 10A-10C, the feeder mechanism 400 includes a cover 402, a stationary body 404, a clamping pivot body 406, a drive input 408, a clamping input 410, a pair of rollers 412, and drive gears 414. FIG. 10A shows the rollers 412 clamped onto an endovascular tool 15 that extends through the feeder mechanism 400.

In the configuration shown in FIG. 10A, rotation of the drive input 408 is transmitted to the rollers 412 via the drive gears 414 such that rotation of the drive input 408 can cause insertion or retraction of the endovascular tool 15. In the illustrated embodiment of the feeder mechanism 400, a single drive input 408 causes rotation of both rollers 412 because rotation of the drive input 408 is transmitted to both rollers 412 by the drive gears 414.

Rotation of the clamping input 410 causes the clamping pivot body 406 to move in and out, thereby transitioning the feeder mechanism 400 between an open configuration (where the rollers 412 are separated allowing the endovascular tool 15 to be loaded into the feeder mechanism 400) and a closed configuration (where the rollers 412 clamp onto the endovascular tool 15). Notably, the feeder mechanism 400 is configured with a top loading slot 416 allowing the endovascular tool 15 to be top loaded into the feeder mechanism 400. This can be advantageous as it is not needed to feed the entire length of the endovascular tool 15 through the feeder mechanism 15 to load it.

FIG. 10B illustrates the pivot body 406 pivoted about pivot point 420 into the open configuration wherein the rollers 412 are separated and the endovascular tool can be loaded into the feeder mechanism.

The cross-sectional view of FIG. 10C provides a view illustrated how the drive input 408 causes rotation of both rollers 412 through a series of drive gears 414. The cross-sectional view of FIG. 10C also shows how the clamping input 410 causes the pivot body 406 to pivot about the pivot 420. Specifically, in the illustrated embodiment, an upper portion of the pivot body 406 is thread ably engaged with a screw mechanism associated with the clamping input 410. The pivot body 406 carries one of the rollers 412 and a drive gear 414.

### Pullwire Reel Mechanisms for Steerable Catheters

The robotic medical systems described herein can be configured for use with quad steerable catheters. Quad steerable catheters are catheters that include, at a distal end thereof, an articulation section that is configured to articulate in four orthogonal directions. In many instances, quad steerable catheters include four pullwires, positioned at 90-degree increments around the longitudinal axis of the catheter. The pullwires can be selectively pulled to cause articulation of the quad steerable catheter. The steerable catheters described herein include reel mechanisms that are configured to be received within a recess 134 of a moveable helm 132. The pullwires of the steerable catheters terminate at a proximal end at the reel mechanism and the reel mechanism is configured to facilitate actuation of the pullwires to cause articulation of the articulation section of the steerable catheter.

As noted, the pullwire reel mechanisms for steerable catheters described herein are configured for use with the robotic medical system 100. The pullwire reel mechanism can be positioned on the proximal end of the catheter. The purpose of the pullwire reel mechanism is to securely hold the catheter and convert the rotary input from the helm's articulation motors into displacements of the four pull wires. The pullwire reel mechanisms can be configured to couple to the four pull wires such that antagonistic pairs (left and right wires or top and bottom, for example) are displaced in equal and opposite directions. In some embodiments, the pullwire reel mechanisms can be sterilized upon assembly and disposed of at the end of the procedure. The reel mechanism can further include an RFID tag that can store the tool's calibration information along with other relevant info of the tool.

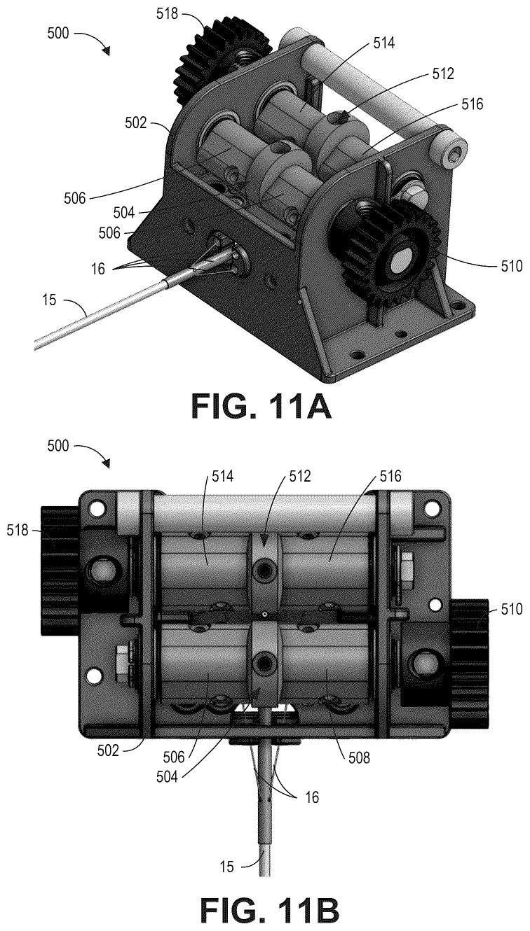

FIGS. 11A and 11B illustrate one embodiment of a pullwire reel mechanism 500 for a quad steerable catheter. The quad steerable catheter can be configured for use with the robotic medical system 100 described herein. During use, the reel mechanism 500 can be received within a recess of a moveable helm 132 such that motors in the moveable helm can actuate the reel mechanism 500 causing articulation of the quad steerable catheter.

In the illustrated embodiment, the pullwire reel mechanism 500 includes a frame 502, a first pulley assembly 504 comprising first a first pulley 506 and a second pulley 508, a first input 510, a second pulley assembly 512 comprising first a first pulley 514 and a second pulley 516, and a second input 518. The elongated body of the catheter 15 extends from the pullwire reel mechanism 500. Pullwires 16 for articulation of a distal portion of the elongated body of the catheter 15 are shown. Although not illustrated in FIGS. 11A and 11B, the pullwire reel mechanism 15 may further include a cover and/or other structures so that it can be removably received within a recess of a helm 132.

As shown, the first input 510 is directly coupled to the first pulley assembly 504 which includes the first pulley 506 and the second pulley 508. Each of these features are directly coupled so that rotation of the first input 510 causes an equal and corresponding rotation of the first pulley 506 and the second pulley 508 of the first pulley assembly. One of an opposing pair of pullwires (e.g., a pullwire associated with upward deflection) is wound on the first pulley 506 in a first direction. The other of the opposing pair of pullwires (e.g., a pullwire associated with upward deflection) is wound on the second pulley 508 in the opposite direction. That is the two pullwires are counter wound. Thus, rotation of the input causes one pullwire to be spooled on the associated pulley, while the other pullwire is unspooled from its associated pulley.

Similarly, the second input 518 is directly coupled to the second pulley assembly 512 which includes the first pulley 514 and the second pulley 516. Each of these features are directly coupled so that rotation of the second input 518 causes an equal and corresponding rotation of the first pulley 514 and the second pulley 516 of the second pulley assembly. One of an opposing pair of pullwires (e.g., a pullwire associated with rightward deflection) is wound on the first pulley 514 in a first direction. The other of the opposing pair of pullwires (e.g., a pullwire associated with left deflection) is wound on the second pulley 516 in the opposite direction. That is the two pullwires are counter wound. Thus, rotation of the input causes one pullwire to be spooled on the associated pulley, while the other pullwire is unspooled from its associated pulley.

When the pullwire reel mechanism 500 is received within the recess 134 of a helm 132, outputs 138 of the helm engage the first and second inputs 510, 518. In this manner, motors in the helm 130 can cause rotation of the first and second inputs 510, 518. The pullwire reel mechanism 500 thus allows for the two outputs 138 of a helm 132 to control four pullwires in a quad steerable catheter.

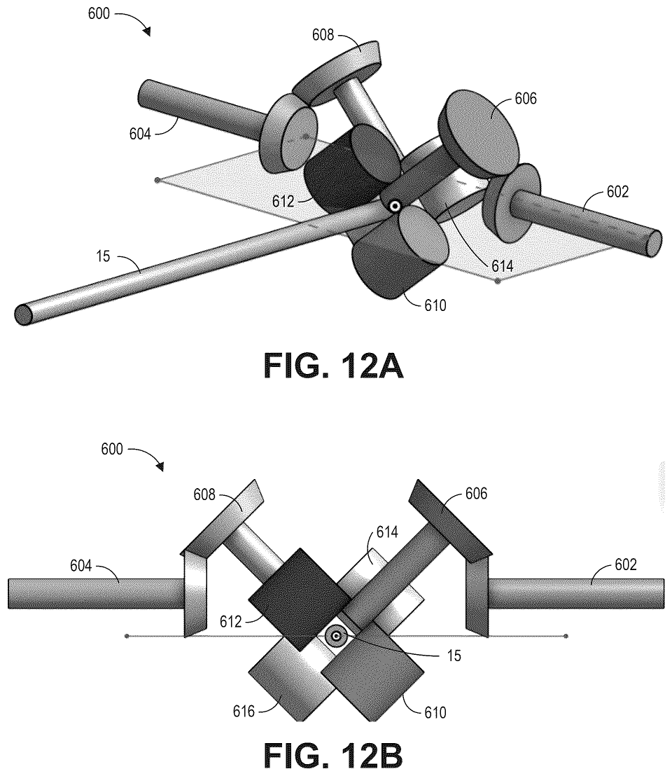

FIGS. 12A and 12B illustrate an example schematic arrangement 600 of inputs 602, 604, drive gears 606, 608, and pulleys 610, 612, 614, 616 for a reel mechanism according to an embodiment. In this arrangement the pulleys 610, 612, 614, 616, are arranged such that a takeoff angle of associated pullwires is minimized. As used herein, the takeoff angle refers to the angle the pullwire at it leaves the elongated body of the endovascular tool to the position at which it first contacts the associated pulley. An advantage of this arrangement is that it minimizes pullwire friction and wear, and aligns the optimal wire path directly out of the catheter exit points. For example, FIG. 12B (a front view), shows how close the innermost edges of each of the pulleys 610, 612, 614, 616 is to the elongated body of the catheter 15. Further in this arrangement 600, each input drives two opposing pulleys by way of an intermediate drive gear. The opposing pulleys can be associated with opposing pullwires on the catheter 15. For example, input 602 (which can be driven by an output 138 of a helm 132) rotates drive gear 606, which is engaged with and drives each of pulleys 610, 612. Similarly, input 604 (which can be driven by an output 138 of a helm 132) rotates drive gear 608 which is engaged with and drives each of pulleys 614, 616.

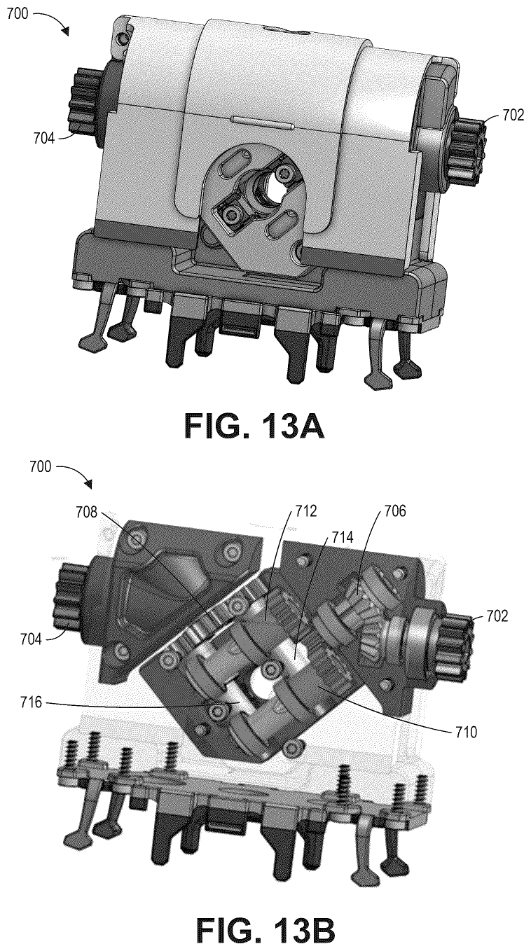

FIGS. 13A and 13B illustrate an embodiment of a pullwire reel mechanism 700 that includes the arrangement 600 shown schematically in FIGS. 12A and 12B. FIG. 13B illustrates the pullwire reel mechanism 700 with a cover thereof shown with transparency to visualize internal components thereof. As shown, an input 702 (which can be driven by an output 138 of a helm 132) rotates drive gear 706, which is engaged with and drives each of pulleys 710, 712. Similarly, input 704 (which can be driven by an output 138 of a helm 132) rotates drive gear 708 which is engaged with and drives each of pulleys 714, 716. The inputs are engaged with the pulleys through a bevel gear.

### Robot-to-Patient Interfaces

The robotic medical systems 100 described herein can include a robot-to-patient (RTP) interface that helps to guide endovascular tools from the stationary helm 130 at the distal end of the endovascular robot 102 to the patient introducer.

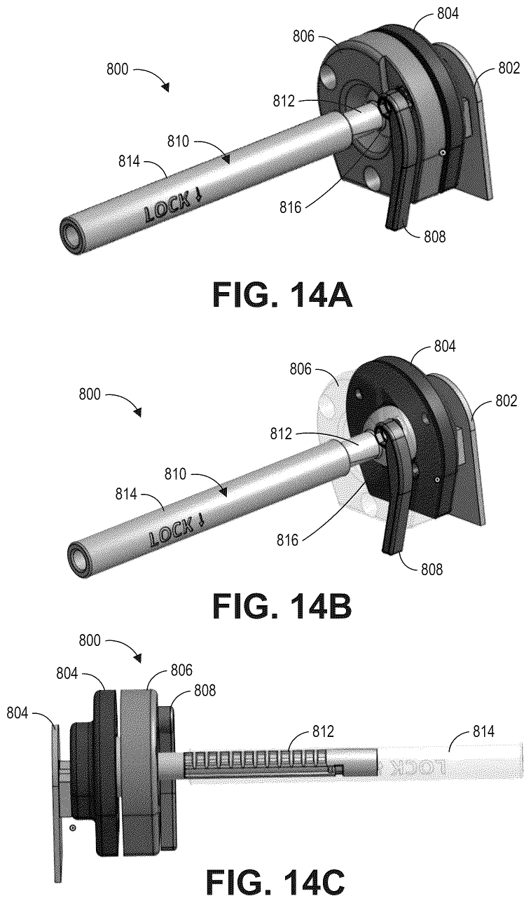

FIGS. 14A-14C illustrate one embodiment of an RTP interface 800. FIG. 14A is a first perspective view, FIG. 14B is a second perspective view with a component shown with transparency, and FIG. 14C is a side view with a component shown with transparency. In the illustrated embodiment, the RTP interface 800 comprises a base plate 802, first and second clamping plates 804, 806, a clamping lever 808, and an extendible tube assembly 810, comprising telescoping first and second tubes 812, 814, and a ball joint 816.

The base plate 802 can be configured to attach to the distal face of the stationary helm 130 or to a feeder mechanism received in the recess 138 of the stationary helm 130. The first clamping plate 804 is fixed to the base plate 802. A ball joint 816 is positioned between the first clamping plate 804 and the second clamping plate 805. A level 808 can include a cam that when the lever 808 is rotated causes the second clamping plate 806 to move toward the first clamping plate 804 to secure the ball joint 816 in place. Accordingly, when the lever 808 is loosened, the position of the ball joint 816 can be adjusted, and when the lever 808 is tightened, the position of the ball joint 816 can be locked in place. An extendible tube assembly 810, comprising first and second telescoping tubes 812, 814 extends from the ball joint 816. The position of the ball joint 816 can be adjusted to adjust an angle of the extendible tube assembly 810. As shown in FIG. 14C, the first tube 812 is received inside the second tube 814. A tooth on the second tube 814 can be received at different locations within the first tube 814 to fix a length of the telescoping tube assembly 810. During use, endovascular tools can pass through a channel formed through the RTP interface 800.

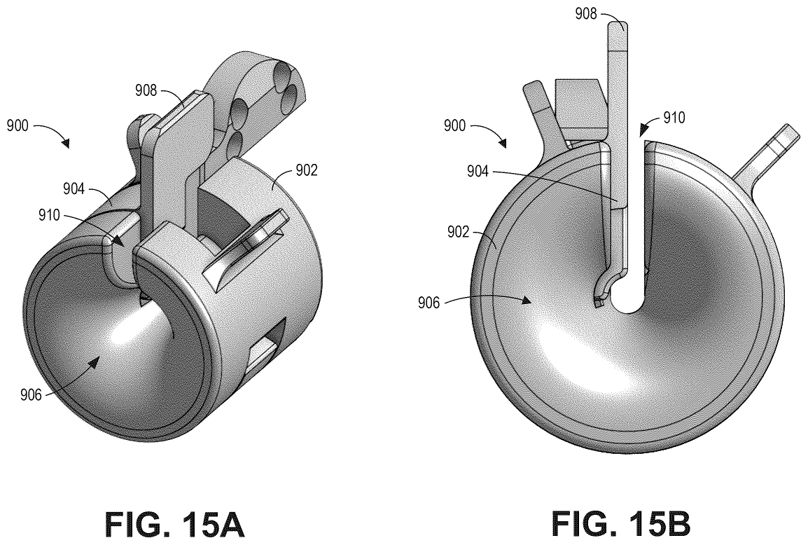

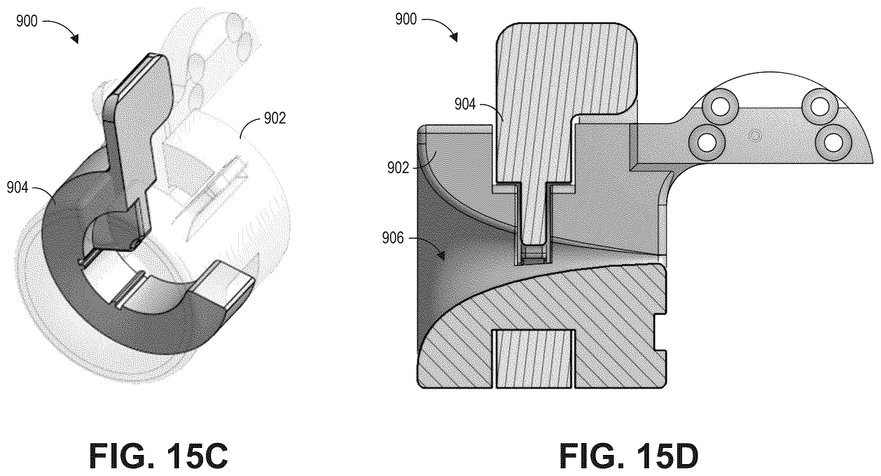

FIGS. 15A-15D illustrate another embodiment of an RTP interface 900. FIG. 15A is a perspective view, FIG. 15B is a front view, FIG. 15C is a perspective view with a component shown with transparency, and FIG. 15D is a cross-sectional view of the RTP interface 900.

As shown, the RTP interface 900 comprises a first body 902 and a second body 904. It the illustrated embodiment, the first body 902 is generally cylindrical. A proximal face of the first body 902 can be configured to attach to the distal face of the stationary helm 130 or to a feeder mechanism received in the recess 138 of the stationary helm 130. A distal face includes a funnel or trumpet like opening 906. The second body 904 can comprise a semicircular body received with a groove formed in the first body 902. The groove of the first body 902 can be circumferentially longer than the circumferential length of the second body 904 to allow the second body 904 to slide with in the groove (see FIG. 15C).

As shown in the FIG. 15B, the second body 904 can slide to apposition to allow access to slot 910 formed in the first body 902. The slot 910 can be used to top load an endovascular tool into the RTP interface 900. With the endovascular tool loaded, the second body 904 can be rotated to close the slot, securing the tool. A tab 908 can be provided on the second body to facilitate rotation.

### Additional Details

It will now be evident to those skilled in the art that there has been described herein methods, systems, and devices for improved routing of catheters and other devices to targeted anatomical locations using robotically controlled assemblies. Although the inventions hereof have been described by way of several embodiments, it will be evident that other adaptations and modifications can be employed without departing from the spirit and scope thereof. The terms and expressions employed herein have been used as terms of description and not of limitation; and thus, there is no intent of excluding equivalents, but on the contrary, it is intended to cover any and all equivalents that may be employed without departing from the spirit and scope of the inventions.

While the disclosure has been described with reference to certain embodiments, it will be understood that various changes may be made, and equivalents may be substituted for elements thereof without departing from the scope of the disclosure. In addition, many modifications will be appreciated to adapt a particular instrument, situation, or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above but should be determined only by a fair reading of the claims that follow.

While the embodiments disclosed herein are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the recited order. The methods disclosed herein include certain actions taken by a practitioner; however, they can also include any third-party instruction of those actions, either expressly or by implication. For example, actions such as “advancing a catheter or microcatheter” or “advancing one portion of the device (e.g., linearly) relative to another portion of the device to rotate the distal end of the device” include instructing advancing a catheter” or “instructing advancing one portion of the device,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

The various illustrative logical blocks, modules, routines, and algorithm steps described in connection with the embodiments disclosed herein can be implemented as electronic hardware, or as a combination of electronic hardware and executable software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a similarity detection system, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A similarity detection system can be or include a microprocessor, but in the alternative, the similarity detection system can be or include a controller, microcontroller, or state machine, combinations of the same, or the like configured to estimate and communicate prediction information. A similar detection system can include electrical circuitry configured to process computer-executable instructions. Although described herein primarily with respect to digital technology, a similarity detection system may also include primarily analog components. For example, some or all of the prediction algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a similarity detection system, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An illustrative storage medium can be coupled to the similarity detection system such that the similarity detection system can read information from, and write information to, the storage medium. In the alternative, the storage medium can be integral to the similarity detection system. The similarity detection system and the storage medium can reside in an ASIC. The ASIC can reside in a user terminal. In the alternative, the similarity detection system and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A feeder mechanism, comprising:
a stationary body;
a pivot body pivotally coupled to the stationary body at a pivot;
a first roller rotatably mounted to the stationary body;
a second roller rotatably mounted to the pivot body;
a first input configured to be rotatable by a corresponding first output on a helm, the first input mechanically coupled to the first roller and the second roller such that rotation of the first input causes rotation of the first roller and the second roller;
a second input configured to be rotatable by a corresponding second output on the helm, the second input mechanically coupled to the pivot body such that rotation of the second input causes the pivot body to rotate about the pivot relative to the stationary body, wherein:
rotation of the second input in a first direction causes the pivot body to pivot to a closed configuration wherein an endovascular tool is clamped between the first roller and the second roller, and
rotation of the second input in a second direction causes the pivot body to pivot to an open configuration wherein the endovascular tool can be loaded into or unloaded from the feeder mechanism.

2. The feeder mechanism of claim 1, further comprising a slot formed in through an upper surface of the feeder mechanism such that the endovascular tool is top loadable into the feeder mechanism.

3. The feeder mechanism of claim 2, wherein the first and second rollers are mechanically coupled to the first input through one or more drive gears.

4. The feeder mechanism of claim of 3, wherein the first and second rollers are rotated in opposite directions.

5. The feeder mechanism of claim 4, wherein the second input is mechanically coupled to the pivot body by a screw mechanism.

6. The feeder mechanism of claim 5, wherein the first input is located on one side of the feeder mechanism and the second input is located on an opposite side of the feeder mechanism.

7. The feeder mechanism of claim 1, wherein the endovascular tool comprises a catheter.

8. The feeder mechanism of claim 1, wherein the endovascular tool comprises a wire.

9. A reel mechanism for a steerable catheter, comprising:
a first input configured to be rotatable by a first output of a helm;
a second input configured to be rotatable a second output of a helm;
a first pulley, a second pulley, a third pulley, and a fourth pulley positioned at 90-degree intervals around an axis, wherein the axis is aligned with an elongated body of an endovascular tool extending from the reel mechanism;
wherein the first input is mechanically coupled to the first pulley and the third pulley, wherein rotation of the first input is configured to cause rotation of the first pulley and the third pulley;
wherein the second input is mechanically coupled to second pulley and the fourth pulley, wherein rotation of the second input is configured to cause rotation of the second pulley and the fourth pulley;
wherein a first pair of opposing pullwires of the elongated body are wound on the first pulley and the third pulley; and
wherein a second pair of opposing pullwires of the elongated body are wound on the second pulley and the fourth pulley.

10. The reel mechanism of claim 9, wherein the first input is mechanically coupled to the first pulley and the second pulley by a first drive gear.

11. The reel mechanism of claim 10, wherein the first drive gear comprises a bevel gear.

12. The reel mechanism of claim 10, wherein the second input is mechanically coupled to the second pulley and the fourth pulley by a second drive gear.

13. The reel mechanism of claim 12, wherein the second drive gear comprises a bevel gear.

14. The reel mechanism of claim 13, wherein the endovascular tool comprises a quad steerable catheter.

15. A robot-to-patient (RTP) interface, comprising:
a first body, the first body comprising a generally cylindrical body extending between a proximal face configured to couple to a helm or a tool positioned in the helm and a distal face, the distal face comprising a funnel-shaped opening, the first body further comprising a longitudinal slot;
a second body comprising a semi-cylindrical body, the second body received within a semi-cylindrical groove formed circumferentially in the first body, the second body slidable within the groove, wherein the second body is slidable between:
a first position wherein the longitudinal slot is open; and
a second position wherein the longitudinal slot is closed.

16. The RTP interface of claim 15, wherein in the open position an endovascular tool can be loaded into the RTP interface such that the endovascular tool extends through the RTP interface.

17. The RTP interface of claim 16, wherein in the closed position the endovascular tool is captured by but is advanceable or retractable through the RTP interface.

18. The RTP interface of claim 17, wherein the second body further comprises a tab extending therefrom.

19. The RTP interface of claim 18, wherein the first body further comprises a tab extending therefrom.

20. The RTP interface of claim 19, wherein the endovascular tool comprises a catheter or a wire.
